(19) United States
(12) Patent Application Publication
(10) Pub. No.: US 2020/0289054 A1
(43) Pub. Date: Sep. 17, 2020

(54) SYSTEM AND METHOD FOR MONITORING BRAIN ACTIVITY USING NEAR- INFRARED SPECTROSCOPY AND SELECTIVELY APPLYING TRANSCRANIAL DIRECT CURRENT STIMULATION AND PHOTOBIOMODULATION THERAPY

(71) Applicant: Mohan Muvvala, Franklin, TN (US)

(72) Inventor: Mohan Muvvala, Franklin, TN (US)

(21) Appl. No.: 16/891,122

(22) Filed: Jun. 3, 2020

Related U.S. Application Data
(60) Provisional application No. 62/857,761, filed on Jun. 5, 2019.

(51) Int. Cl. A61B 5/00; A61N 5/06

(52) U.S. Cl. CPC A61B 5/4836; A61B 5/4064; A61B 5/6803; A61B 5/0082; A61B 5/7264; A61B 5/0022; A61N 2005/0632; A61N 5/0618; A61B 2560/0204; A61N 2005/0651; A61N 2005/0659; A61N 2005/0626; A61N 2005/0647; A61B 5/0075

(57) ABSTRACT

Presented is a non-invasive and non-medication treatment system for patients suffering from mental dysfunction or disorder such as anxiety, depression and chronic pain. The system uses near-infrared spectroscopy (NIRS) sensors for detecting or monitoring brain activity of a user/patient, and then subject to detecting a low brain activity area in a patient's brain, facilitates a patient to apply Photobiomodulation Therapy (PBMT) and/or transcranial Direct Current Stimulation (tDCS) to treat the brain dysfunction. The simulation procedure adapted by the patient is either auto chosen by a program product embodied in the patient device having machine learning and artificial intelligence capabilities or manually fed by the patient over the patient device.

### CROSS-REFERENCE TO RELATED PATENT DOCUMENTS

This patent application claims the benefit of priority of U.S. Provisional Application No. 62/857,761 entitled “BRAIN STIMULATION SYSTEM AND METHOD,” filed Jun. 5, 2019, which is hereby incorporated herein by reference in its entirety.

### TECHNICAL FIELD

The present invention relates generally to systems and methods for treating brain dysfunction or disorder and, more specifically, to a system and method that uses near-infrared spectroscopy (NIRS) sensors for detecting brain activity of a user/patient, and then subject to detecting a low brain activity area in the patient's brain, facilitate the user with applying Photobiomodulation Therapy (PBMT) and/or transcranial Direct Current Stimulation (tDCS).

### BACKGROUND

Millions of people in the world are affected by different mental health conditions every year. In the US alone, according to a report published by National Institute of Mental Health (NIMH) approximately 1 in 5 adults experiences mental illness in any given year. There could be various reasons to it. Some of the reasons could be anxiety, specific phobia, post-traumatic stress disorder, agitation, increased appetite and weight gain, insomnia, and so on. A report from NIMH, revealed that about 31.1% of U.S. adults experience anxiety disorder at some time in their lives. A report published in 2009 by Healthcare-Cost and Utilization Project (H-CUP) revealed that mental disorders, including major depression, dysthymic disorder and bipolar disorder, are third most common cause of hospitalization in the U.S. for both youth and adults aged in between 18-44.

Medication and hospitalization are common types of treatment available for the people suffering from mental disorder or dysfunction. The medication that are commonly given to patients suffering from anxiety, and depression are antidepressants, and anti-anxiety medication often in combination with psychotherapy. These medication may be able to provide a temporary relief to the patient, however in long run, these medication cause a sever health problems to the patients. Further, in another form of treatment, the patients are hospitalized where they are closely monitored, and then diagnosed with invasive treatments such as Neuro-surgery. Patients undergoing such procedure are often open to several risks related to invasive surgeries such as brain stroke, coma, brain swelling, and blood clotting in the brain and so on.

Different types of brain stimulation therapies are best alternatives to these conventional treatments for brain dysfunction or disorder. One of the commonly known brain stimulation therapy includes Photobiomodulation Therapy (PBMT) which is a form of light therapy that leverages non-ionizing light sources including lasers, light emitting diodes, and/or broadband light, in the visible (400-700 nm) and near-infrared (700-1100 nm) electromagnetic spectrum. Another most commonly used brain stimulation therapy includes transcranial Direct Current Stimulation (tDCS) which is a form of neuro-stimulation that utilizes constant, low direct current delivered via electrodes placed on the patient's head. Yet another commonly used brain stimulation therapy includes Transcranial Magnetic Stimulation (TMS) that uses magnetic fields to stimulate nerve cells in the brain to improve symptoms of depression.

All of these stimulation mechanisms are non-invasive procedure and do not require any medication and yet able to provide better results with no side effects over the conventional medicinal and invasive procedures adapted for use by patients to treat mental disorder such as depression, anxiety and chronic pain.

Some of prior art literature discloses various systems and methods using one or more of these stimulation procedures for treatment of brain dysfunction. Such as for example: U.S. Pat. No. 9,456,784 discloses a Transcranial Magnetic Stimulation (TMS) apparatus for providing TMS to a patient. The patent discloses a head mount for disposition on the head of the patient, and a plurality of magnet assemblies for releasable mounting on the head mount, wherein each of the magnet assemblies comprises a magnet for selectively providing a rapidly changing magnetic field capable of inducing weak electric currents in the brain of a patient so as to modify the natural electrical activity of the brain of the patient.

WO2018213722 discloses sequential application of all of those above discussed stimulation procedures (PBMT, tDCS, TMS) to treat dysfunction of the brain. Specifically, the patent application discloses use of electroencephalography (EEG) for recording neural activity of selected brain region of the patient where the treatment has to be applied. Using the electrodes the EEG data is first recorded which is then analysed to identify an area of high and low activity in the brain and then a sequence of transcranial stimulations are provided to treat the low activity area of the patient's brain.

US20130079659 discloses an apparatus that integrates tDCS and EEG to electrodes that are placed on the user head. As disclosed in the patent application, the same set of electrodes are used for both reading brain activity (EEG data) and applying tDCS simulation, A switching arrangement is further used to selectively connect either a tDCS drive arrangement or an EEG analyzer to the electrodes. A digital controller is coupled to control the switching arrangement via a bus. The tDCS drive arrangement includes independent constant current sources. A digital-to-analog converter (DAC) is coupled to control the constant current sources, and the digital controller is coupled to control the DAC via the bus. A host computer controls the digital controller via a host interface. The host computer provides a user interface that receives input from a user and provides output information to the user.

Although solutions are provided in the past that make use of various brain stimulation procedures and processes for monitoring brain disorder/dysfunction, there still remains a need for a more reliable solution especially for monitoring the brain signals/neuro signals in order to appropriately identify the dysfunctional area of brain to apply the transcranial direct current stimulation and/or photobiomodulation therapy to treat the identified dysfunctional area of the patient's brain.

### SUMMARY

It is an objective of the present invention to provide a non-invasive and non-medication treatment to users suffering from brain dysfunction or disorder such as anxiety, depression and chronic pain.

It is another objective of the present invention to provide a treatment system and method for brain dysfunction that patients can self-administer at home without having to visit healthcare facilities.

It is another objective of the present invention to provide a treatment system and method for brain dysfunction that when applied provides short term and long term lasting effects in the patients, thus requiring less frequent application of stimulation therapies again.

It is another objective of the present invention to provide a treatment system and method for brain dysfunction that's highly effective in maximizing neuroplasticity, optimizing neural pathways, amplifying production of primary source of energy production i.e. adenosine triphosphate (ATP), increasing signalling molecule (nitric oxide) that is widely used in a nervous system, reducing neuro-inflammation, increasing and/or improving cellular lifespan and health, cellular proliferation and migration.

Another object of the present invention is to provide a treatment system and method for brain dysfunction which is enabled by artificial intelligence and machine learning capabilities because of which the proposed system is able to predict possible recommendations on treatment to the user/patient.

Embodiments of the present invention disclose a brain activity monitoring and stimulation system. The system includes a head mount configured to be placed on a patient's head to cover the patient's head or brain fully, or to cover at least a portion of the patient's brain, the head mount comprising: one or more Near-Infrared Spectroscopy (NIRS) sensors in electrical communication with one or more near-infrared LEDs, the one or more NIRS sensors are configured to detect brain activity from a selected tissue site located on the patient's brain using the one or more near-infrared LEDs, wherein the one or more near-infrared LEDs are signalled by the one or more NIRS sensors to deliver near-infrared light or energy to the selected tissue site located on the patient's brain to capture the brain activity.

According to the embodiment, the system further includes a microcontroller in electrical communication with the one or more NIRS sensors, a Photobiomodulation Therapy (PBMT) unit, a transcranial Direct Current Stimulation (tDCS) unit, and a patient device (104) associated with a patient (106), the microcontroller (105) is configured for: receiving the detected brain activity from the one or more NIRS sensors (114), relaying the detected brain activity to the patient device (104); triggering at least one of: the Photobiomodulation Therapy (PBMT) unit, and the transcranial Direct Current Stimulation (tDCS) unit to apply a selected simulation procedure at the selected tissue site located on the patient's brain in order to treat dysfunction of the selected tissue site.

According to the embodiment, the selected simulation procedure being applied is received by the microcontroller from the patient device, wherein the selected simulation procedure is either auto suggested by a program product having an artificial and machine learning module and installed on the patient device, or manually configured by the patient.

These and other features and advantages of the present invention will become apparent from the detailed description below, in light of the accompanying drawings.

### BRIEF DESCRIPTION OF THE DRAWINGS

FIG. 1 illustrates a general environmental scenario showing use of proposed brain activity monitoring and stimulation system of the present invention, in accordance with an exemplary embodiment;

FIG. 2 illustrates a block diagram representation of the proposed brain activity monitoring and stimulation system of the present invention, in accordance with an exemplary embodiment;

FIG. 3 illustrates a general schematic circuit diagram representation of components operationally and electrically configured in a head mount of the proposed brain activity monitoring and stimulation system of the present invention, in accordance with an exemplary embodiment; and

FIG. 4 illustrates a general computing environment of a user's device enabled by artificial intelligence and machine learning.

### DETAILED DESCRIPTION

Before describing the present invention in detail, it should be observed that the present invention utilizes a combination of components or processes, which constitutes a system and method for monitoring brain activity of the user's brain and in response to detecting brain activity (low activity area) of the user's brain selectively applying transcranial direct current stimulation and/or photobiomodulation therapy. Accordingly, the components or processes have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific component level details and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items. Further, for the purpose of this invention, the terms “user”, and “patient” are interchangeably used and basically refer to a person using the proposed invention for treating brain dysfunctions such as anxiety, depression and chronic pain.

As used herein, the terms “depression,”, “anxiety,” and “chronic pain,” may refer to a condition or disorder, which can range from being mild to severe, that negatively affects how people feel, the way they think and how they act. These conditions may cause feelings of sadness and/or a loss of interest in activities once enjoyed by a user/patient. Depression and anxiety symptoms can vary from mild to severe and can include: feeling sad or having a depressed mood, loss of interest or pleasure in activities once enjoyed, changes in appetite—weight loss or gain unrelated to dieting, trouble sleeping or sleeping too much, loss of energy or increased fatigue, slowed movements and speech (actions observable by others), feeling worthless or guilty, difficulty thinking, concentrating or making decisions and thoughts of death or suicide.

Further, as used herein, the term “treatment,” “therapy,” “treating,” etc are refer to an action upon a disease, disorder, or condition such as depression, anxiety, or chronic pain to reduce or ameliorate harmful or any other undesired effects of these disease, disorder, dysfunctions or condition. “Treatment,” or “therapy” as used herein, covers the treatment of the patient/user in need thereof, by taking steps to obtain beneficial or desired results in the patients/users, preventing the disorder from reoccurring or at least providing a short term or long term relief or reducing the development of the disease.

The term “tissue site” for the purpose of this invention means the brain area (neural area) located underside the near infrared LEDs or the electrodes positioned in the head gear/head mount, when the head gear or head mount is worn by the patient/user for monitoring the brain activities and treating any brain dysfunction.

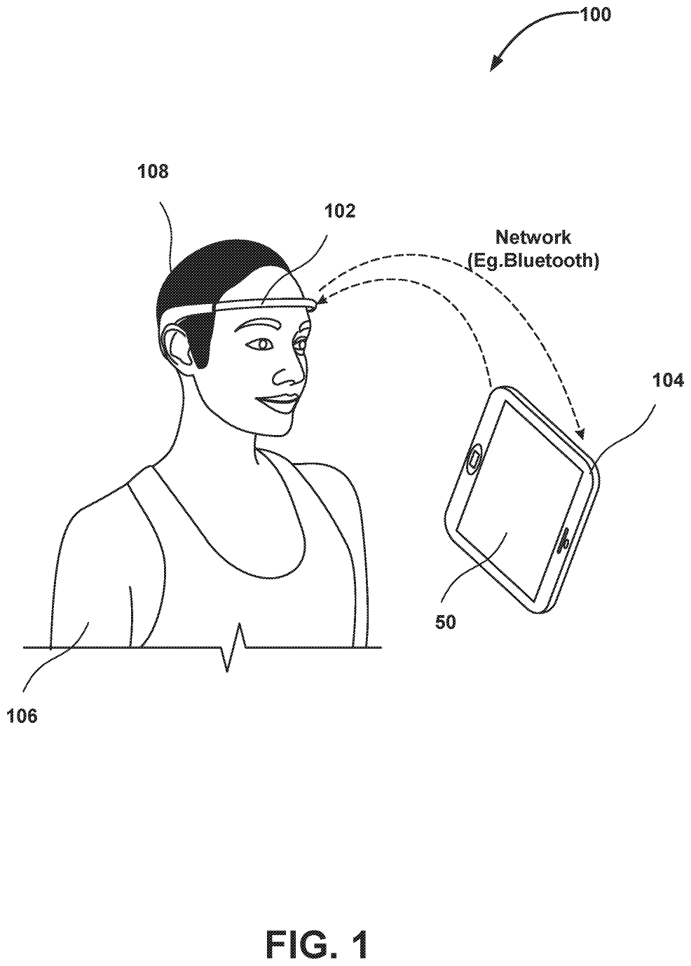

The proposed system and method for monitoring neural images or brain activities in a patient having brain dysfunction and treating the same by applying transcranial direct current stimulation and/or Photobiomodulation therapy will now be described with reference to the accompanying drawings, particularly FIGS. 1-4.

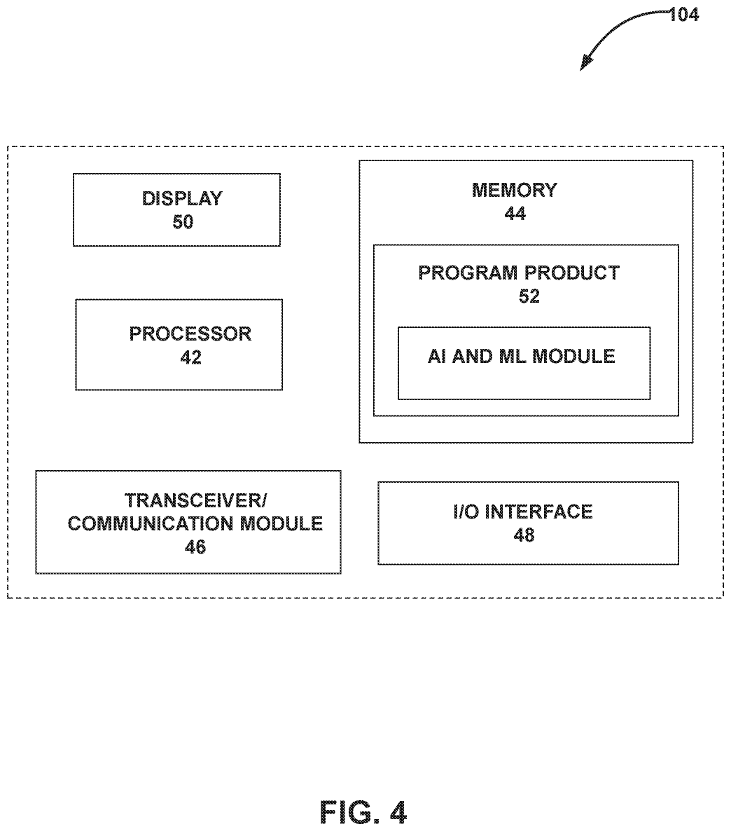

Referring to accompanying figures, particularly to FIG. 1, the proposed brain activity monitoring and stimulation system 100 of the present invention includes a head mount 102 configured to be placed on a user's head 108 to cover the user's head 108 fully, or to cover at least a portion of the user's brain being treated such as a forehead. In the example shown, the head mount 102 is configured in the form of a head band to cover portion of the user's head such as forehead region or frontal lobe region, cerebral cortex region, and part of cerebellum. The head mount 102 may be configured in many other forms but are not limited to a helmet like head gear that would substantially cover the user's head in its entirety. The system 100 further includes a user device/a patient's device 104 associated with the user or patient 106 undergoing treatment using the proposed system 100. The proposed system 100 is so designed that the user/patient 106 can self-use the system 100 with ease without having to visit any healthcare facilities. The patient's device 104 preferably includes but not limited to a smart phone, a tablet, a laptop, and so on. The device 104 has all general capabilities that any communication or computing devices usually have as shown and described with respect to FIG. 4. The device 104 is configured to communicate with the head mount 102 over a network preferably but not limited to Bluetooth, WiFi, and so on. Additionally, the device 104 is further configured with a program product that embeds the device 104 with artificial intelligence and machine learning capabilities.

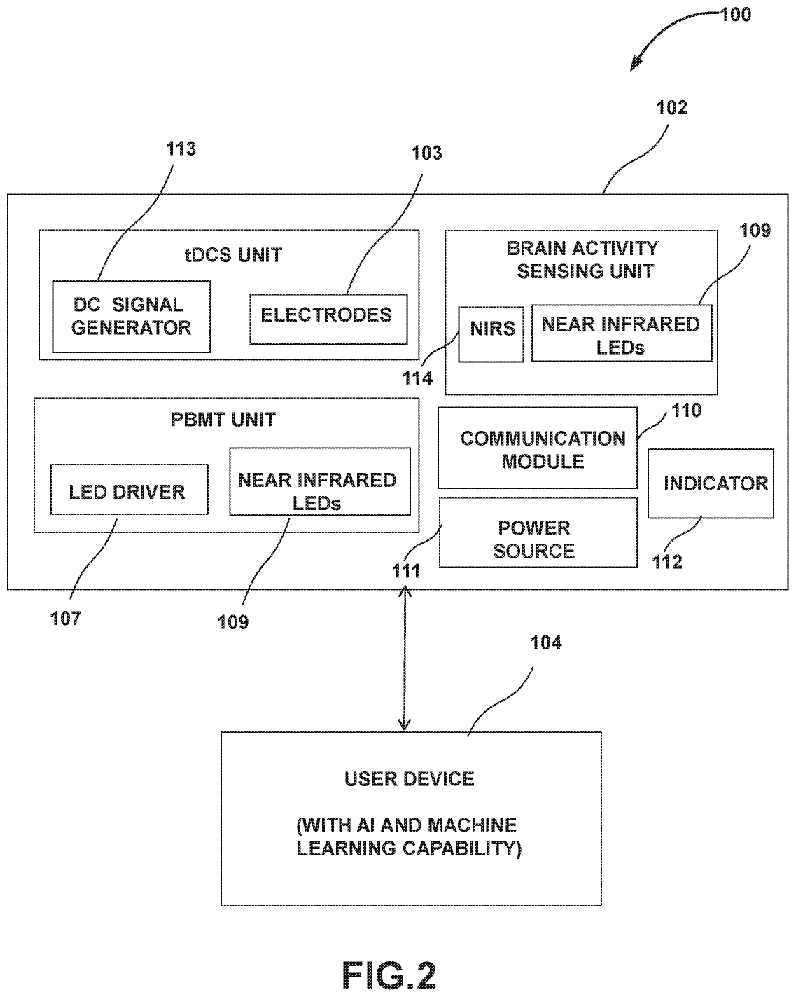

Referring to FIG. 2, a more detailed block diagram representation of the proposed brain activity monitoring and stimulation system of the present invention is shown. As seen, the head mount 102 includes an indicator 112, a brain activity sensing unit consisting of one or more Near-Infrared Spectroscopy (NIRS) sensors 114, and one or more near Infrared LEDs 109. The brain activity sensing unit is configured to sense or detect the brain activities of the user's brain, particularly the region of brain laid directly underneath the near Infrared LEDs 109 disposed in the head mount 102, a communication module 110 for relaying the detected brain activities by the NIRS sensors 114 to the user/patient device 104 loaded with the program product. The head mount 102 further includes transcranial direct current stimulation (tDCS) unit consisting of electrodes 103, and DC signal generator 113 for applying a constant transcranial direct current to the low brain activity area of the brain for treating the dysfunction of the low activity brain area, a Photobiomodulation therapy (PBMT) unit that makes use of the near infrared LEDs 109 driven by LED driver to simulate and treat the low activity area of brain or dysfunctional area of brain with near infrared light, and a power source 111 for powering the tDCS unit, the PBMT unit, brain activity sensing unit, the indicator 112, and the communication module 110. The electrodes are available in pair forms, a first electrode (anode) 103a, and a second electrode (cathode) 103b. The head mount 102 may have multiple electrodes strategically disposed over the head mount 102 so that when the head mount 102 is worn, the electrodes 103 come in contact with the user's scalp. The head mount 102 may be featured to have some straps or tapes or other mechanisms that may help the users to precisely adhere the electrodes 103 to their scalp while wearing the head mount 102.

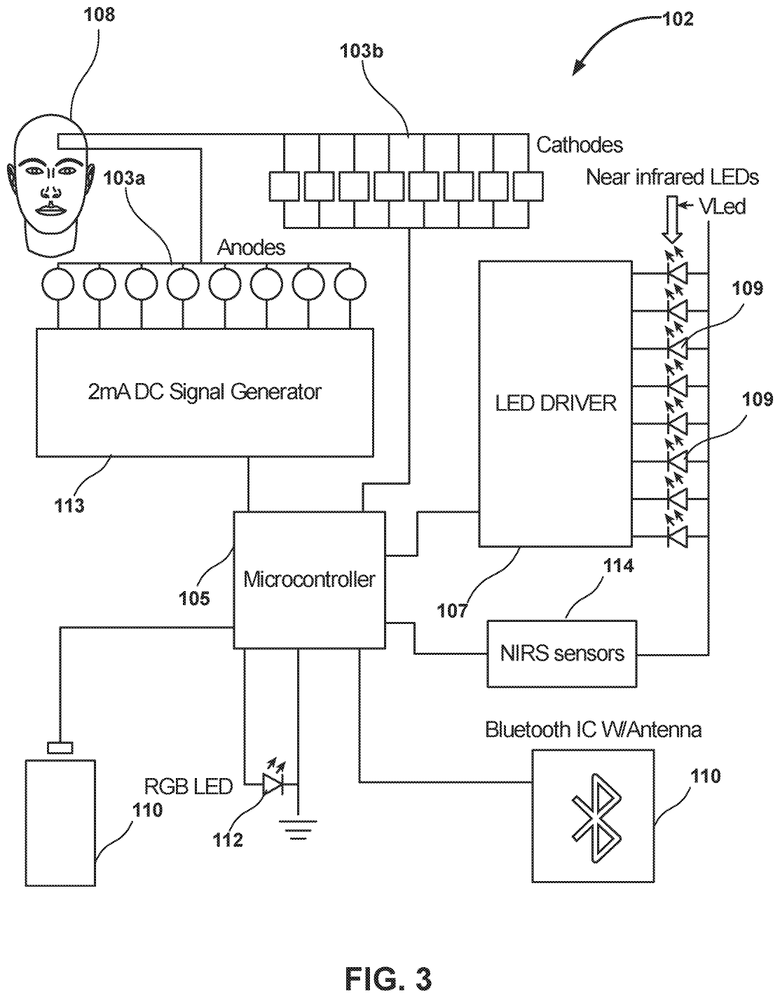

Referring to FIG. 3, a general schematic circuit diagram representation of components operationally and electrically configured in the head mount 102 of the brain activity monitoring and stimulation system 100 is shown.

As discussed, the head mount 102 includes one or more Near-Infrared Spectroscopy (NIRS) sensors 114 in electrical communication with one or more near infrared LEDs 109. The NIRS sensors 114 are configured to detect brain activity from a selected tissue site located on a patient's brain. When the head mount 102 is mounted over the user/patient's head 108, the near infrared LEDs 109 disposed in the head mount 102 come in contact or near to the brain region (specific tissue site) that's being monitored or treated. The NIRS sensors 114 are able to detect the brain activity or neural images from the selected tissue site of the brain located on the patient's brain using these near-infrared LEDs 109. The near infrared LEDs 109 are configured to deliver near-infrared light or energy to the selected tissue site located on the patient's brain to capture the brain activity from the selected tissue site upon receiving the signal from the NIRS sensors 114. The near infrared LEDs operate at near-infrared wavelengths of 780 nm to 2500 nm.

The head mount 102 further includes a microcontroller 105 in electrical communication with the NIRS sensors 114. The microcontroller 105 may comprise embedded logics for receiving, and processing the detected brain activity from the NIRS sensors and command/input from the user device 104. The term “embedded logics” used herein is used in broader sense that include programs, or routines, or interfaces stored in a memory that the microcontroller 105 can fetch and execute to perform the function of receiving and processing of the detected brain activity from the NIRS sensors 114 and command/instruction inputted or selected by the user 106. Once the microcontroller 105 receives the detected brain activity from the NIRS sensors 114 and processes the same, the microcontroller 105 relays the detected brain activity information to the user/patient 106 over the user device 104 having the program product installed therein.

The program product configured at the user device 104 includes an artificial intelligence (AI) and machine learning (ML) module that helps the user device 104 perform decision making function. Due to being AI and ML enabled, the user device 104 upon detecting the pattern of the brain activity of the user's brain compares the brain activity with historical brain activity data related to the user 106 and historical stimulation procedures received by the user/patient 106. Upon learning the latest brain activity and past historical brain activity data, and the simulation received by the user/patient 106 in the past, the program product is able to predict the possible stimulation procedures (tDCS or PBMT or both), specific doses for the predicated stimulation procedures that the user should possibly intake, or any other procedures or actions that the user/patient 106 should follow as a course of treatment for brain dysfunction or disorder.

For example, let's assume a user/patient 106 is anxious and assuming the head mount 102 (as shown in FIG. 1) is worn by the user with the near infrared LEDs 109 and electrodes 103 laid in contact or in proximity to the user's frontal lobe region and the cerebral cortex region at the back. Upon detecting the brain activity or neural imaging (by the NIRS sensors 114) in these brain regions, the detected brain activity or neural images are passed (by the microcontroller 105) to the user device 104 having the program product. The program product is able to suggest or predict possible actions that would benefit the user/patient 106 in the treatment of brain dysfunction after analysing the detected brain activity of the user. According to the embodiment, the program product empowers the user device 104 with the artificial and machine learning capability due to which upon receiving the detected brain activity, the program product facilitates the user to view the same and the program product further compares the detected brain activity with the historical or past brain activities and the treatments taken by the user 106. Upon comparing, the program product is able to convey one or more messages or information (via Graphical User interface (GUI) or by push notifications etc) to the user 106 on the possible treatment that the user 106 can follow. In the example being discussed where the user is anxious, if it is found that the frontal lobe of the user/patient's brain have lesser blood flow, which is usually the case (when the person is anxious the medulla and cerebral cortex region of brain usually have high brain activity and the frontal lobe have low activity), then the program product would may suggest applying tDCS, and suggest the user 106 to activate the electrode 103a (anode) in the frontal lobe area so the current flows through the frontal lobe to the area of high activity of brain i.e cerebral cortex where the electrode 103b (cathode) is placed. This treatment using tDCS usually provides short term relief to the patient 106.

Once the user/patient 106 inputs his/her choice (on suggested stimulation procedures, doses or other suggested actions over the GUI), the selected treatment or simulation procedure is then relayed to the microcontroller 105 over the Bluetooth network. The microcontroller 105 then triggers Photobiomodulation Therapy (PBMT) stimulation or transcranial Direct Current Stimulation (tDCS) or both, as the requirement may be depending upon the selected simulation procedure to simulate the selected tissue site located on the patient's brain to treat dysfunction of the selected tissue site. In particular, when applied these stimulation procedures maximizes neuroplasticity in the brain region where the procedures are applied or restructures the neural pathways in the brain region where the stimulation procedures are applied. In the preferred embodiment, the user/patient 106 selects the suggested simulation procedure from the program product. In another embodiment, the user 106 is able to manually configure the simulation procedure himself/herself by observing the brain activity. The simulation procedure in the context of present invention refers to:
1. selecting appropriate simulation method, namely tDCS or PBMT or both, and
2. selecting simulation doses associated with the selected simulation method, for example, setting current level (say for example 2 mA, 3 mA, 4 mA) and time duration (say 10 minutes, 20 minutes) in case tDCS simulation is selected, similarly setting the near infrared wavelength or light intensity and time duration in case PBMT is selected.

In order to provide the tDCS, the head mount 102 is provided with the electrode 103a (anode), and the electrode 103b (cathode), both in electrical communication with the microcontroller 105 (there may be multiple anode 103a and cathode terminals 103b located at the selected tissue site). The electrode 103a is further connected to the micro controller 105 through an intermediate DC signal generator 113. When the microcontroller 105 receives the command/input or selected simulation procedure from the user 106, the microcontroller 105 ensures a low DC current flow from the terminal (anode terminal) 103a to the terminal (cathode) 103b. In an example, this low current can range from 1-4 mA depending upon the severity of the brain dysfunction. In order to ensure, only a small magnitude of current flows from the anode to the cathode, the low current signal generator 113 (such as 1 mA or 1.5 mA or 2 mA, or 3 mA, or 4 mA current generator) is disposed between the microcontroller 105 and the anode terminal 103a to act upon supplied current or voltage from the microcontroller 105, which is then converted to a constant or nearly constant low DC current (of 2 mA for example since 2 mA signal generator is shown used in FIG. 3) that can then be given to the patient's brain at the dysfunctional tissue site. This low level current is passed directly through skin/scalp and skull of the user/patient 106 to selected tissue site located on the patient's brain where stimulation need to be applied. Upon applying the tDCS, overall neuronal activity of the user's brain changes due to collective effects. Effectiveness of the tDCS is function of the intensity and duration of the tDCS. Based on the detected neural images or brain activity, the system 100 is able to predict the suitable dose of the tDCS that the patient/user 106 should be using for proper treatment. In an example, the patient/user 106 may be suggested to use the tDCS for 20 minutes with intensity of current as 2 mA.

In order to provide the PBMT therapy, the head mount 102 makes use of the near infrared LEDs 109. The near infrared LEDs 109 operate at near-infrared wavelengths of 780 nm to 2500 nm. When the microcontroller 105 receives the command/input or selected simulation procedure from the user 106 over the Bluetooth or the like network (when the user enters or selects the suggested simulation procedure over the GUI on the user device 104), the microcontroller 105 signals the LED driver 107 to activate the near infrared LEDs 109 for certain time (as per the simulation procedure) and allow the near infrared LEDs 109 to pass the infrared light or energy to the selected tissue site located on the patient's brain in order to stimulate the selected tissue site. In an example, the near infrared LEDs 109 may be activated for say 20 minutes for stimulating the effected tissue site chosen for treatment.

Further, according to the embodiment, the microcontroller 105 is further configured to automatically switch between detecting or monitoring mode when the brain activity (neural images) of the selected tissue area are captured, and the simulation mode when the simulation is applied for treatment. This switching action helps in continuously checking/monitoring brain activity and tracking the effect of the stimulation procedure used or applied on the user's dysfunctional part of the brain. The switching action of the system 100 between the brain activity monitoring mode and the simulation mode is quick (in preferably micro seconds) so as not to obstruct the ongoing simulation procedure or treatment the user 106 is undergoing.

According to the embodiment as seen in FIG. 3, the head mount 102 further includes a visible light indicator 112 such as for example a RGB LED. The visible light indicator 112 is preferably configured to provide an indication if the head mount 102 is in ON state or OFF state. The visible light indicator 112 when not glowing indicates that the head mount 102 is in the OFF state.

According to the embodiment as seen in FIG. 3, the head mount 102 further includes a power source 111 configured for powering the microcontroller 105 and other components (such as the signal generator 113 or the electrodes 103, the LED driver 107, the NIRS sensors 114, and so on) in electrical communication with the microcontroller 105.

According to the embodiment as seen in FIG. 3, the head mount 102 further includes a communication module 110 in communication with the microcontroller 105 for relaying information or messages or instructions to the patient/user 106 over the user/patient's device 104 having the program product configured thereon. In the embodiment, the communication module 110 comprises a Bluetooth module. The relayed information may be related to brain activity. The information that may be received is preferably related to the simulation procedure that the user/patient 106 should follow for treating the brain dysfunction.

In accordance with an example implementation, as shown in FIG. 4, the user/patient's device 104 may include at least one or more processors, such as a processor 42, one or more memory, such as memory 44, a transceiver or communication module 46, one or more I/O interfaces, such as an I/O interface 48, a display 50, a program product 52 having an AI and machine learning module stored in the memory 44.

The processor 42 may be communicably coupled with the transceiver/communication module 46 to receive signals. Further, the transceiver 46 may be configured to transmit signals generated by the processor 42. The processor 42 is in communication with the memory 44, wherein the memory 44 includes the program product 52 with artificial and machine learning module to embed artificial and machine learning capabilities in the user device 104 as described above with respect to FIGS. 1, 2, and 3. The program product 52 and the AI and ML module is configured in the form of routines, programs, objects, components, data structures and the like, which perform particular tasks to be executed by the processor 42. The user/patient's device 104 may be connected to other information processing devices by using the I/O interface 48. The other information processing devices may be stand alone servers remotely located or servers of other healthcare facilities that may require the patient related data (such as brain activity related data or information, simulation procedure related information) for updating the patient related Electronic medical records (EMR). The display 50 of the user device 104 may be utilized to receive inputs from the user/patient 106. The display 50 of the user device 104 may be utilized to provide information or messages or render GUIs generated by the program product to the user/patient 106. The I/O interfaces 48 may include a variety of software and hardware interfaces, for instance, interface for peripheral device(s) such as a keyboard, a mouse, a scanner, an external memory, a printer and the like. In an embodiment, the processor 42 may include different types of processors known in the art including neural network-based algorithms that are effectively used in several applications. In an aspect of the present invention, processor or the neural network may process large amount of data in real-time.

As described above the user/patient's device 104 may include but not limited to a smart phone, a tablet, a desktop computer, a laptop computer, a workstation, a personal digital assistant (PDA) or other device that may communicate data via the network and may display information to the users using the proposed brain activity monitoring and simulation system using GUIs. Examples of types of the network includes but are not limited to a Local Area Network (LAN), a wide area network, a radio network, a virtual private network, an internet area network, Bluetooth, Near field communication, a metropolitan area network, a satellite network, Wi-Fi, a wireless network, GPRS, 2G/3G/4G, and a telecommunication network.

Although particular embodiments of the invention have been described in detail for purposes of illustration, various modifications and enhancements may be made without departing from the spirit and scope of the invention.

What is claimed is:

1. A brain activity monitoring and stimulation system (100), comprising:
a head mount (102) configured to be placed on a patient's head (108) to cover the patient's head or brain fully, or to cover at least a portion of the patient's brain, the head mount (102) comprising:
a brain activity sensing unit having one or more Near-Infrared Spectroscopy (NIRS) sensors (114) in electrical communication with one or more near-infrared LEDs (109), the one or more NIRS sensors (114) are configured to detect brain activity from a selected tissue site located on the patient's brain using the one or more near-infrared LEDs (109), wherein the one or more near-infrared LEDs (109) are signalled by the one or more NIRS sensors (114) to deliver near-infrared light or energy to the selected tissue site located on the patient's brain to capture the brain activity;
a microcontroller (105) in electrical communication with the one or more NIRS sensors (114), a Photobiomodulation Therapy (PBMT) unit, a transcranial Direct Current Stimulation (tDCS) unit, and a patient device (104) associated with a patient (106), the microcontroller (105) is configured for:
receiving the detected brain activity from the one or more NIRS sensors (114),
relaying the detected brain activity to the patient device (104), and
triggering at least one of: the Photobiomodulation Therapy (PBMT) unit, and the transcranial Direct Current Stimulation (tDCS) unit to apply a selected simulation procedure at the selected tissue site located on the patient's brain in order to treat dysfunction of the selected tissue site; and
Wherein, the selected simulation procedure being applied is received by the microcontroller (105) from the patient device (104), wherein the selected simulation procedure is either auto suggested by a program product having an artificial and machine learning module and installed on the patient device (104), or manually configured by the patient (106).

2. The system (100) of claim 1, wherein the Photobiomodulation Therapy (PBMT) unit comprising a LED driver (107) in electrical communication with the one or more near-infrared LEDs (109) for electrically driving the near-infrared LEDs (109) to deliver near-infrared light or energy to the selected tissue site located on the patient's brain for simulating the selected tissue site for treating brain dysfunction.

3. The system (100) of claim 1, wherein the transcranial Direct Current Stimulation (tDCS) unit comprising a first electrode (103a), and a second electrode (103b) configured on the head mount (102) and in electrical communication with the microcontroller (105), and wherein the first electrode (103a) is connected to the micro controller (105) through an intermediate DC signal generator (113).

4. The system (100) of claim 3, wherein the transcranial Direct Current Stimulation (tDCS) unit when triggered by the microcontroller (105) generates a low power DC current that flows through the first electrode (103a) to the second electrode (103b).

5. The system (100) of claim 1, wherein the selected simulation procedure comprising:
a simulation method selected from at least a transcranial Direct Current Stimulation (tDCS) and a Photobiomodulation Therapy (PBMT), and
doses associated with the selected simulation method.

6. The system (100) of claim 5, Wherein the doses associated with the transcranial Direct Current Stimulation (tDCS) comprising magnitude of current that should be applied to the selected tissue site being treated, and a time duration for which the current should be applied at the selected tissue site.

7. The system (100) of claim 5, Wherein the doses associated with the Photobiomodulation Therapy (PBMT) comprising selecting the near infrared wavelength or intensity that should be applied to the selected tissue site being treated and a time duration for which the current should be applied at the selected tissue site.

8. The system (100) of claim 1, wherein the head mount (102) further comprising a visible light indicator (112) configured to provide an indication if the head mount 102 is in ON state or OFF state.

9. The system (100) of claim 1, wherein the head mount (102) further comprises a power source (111) for powering the head mount (102).

10. The system (100) of claim 1, wherein the head mount (102) further comprising a communication module (110) configured thereon and in communication with the microcontroller (105) for relaying the detected brain activity from the selected tissue site located on the patient's brain to the patient (106) over the patient's device (104), and receiving the selected simulation procedure from the patient (106) inputted or selected by the patient (106).

11. The system (100) of claim 10, wherein the communication module (110) comprises a Bluetooth module.

12. The system (100) of claim 1, wherein the near infrared LEDs (109) operate at near-infrared wavelengths of 780 nm to 2500 nm.

13. The system (100) of claim 1, wherein the selected simulation procedure is auto suggested by the program product having the artificial and machine learning module by comparing the detected brain activity detected by the NIRS sensor (114) with historical brain activity data related to the patient (106) and historical stimulation procedure related data received by the patient (106).
